(19) United States
(12) Patent Application Publication
(10) Pub. No.: US 2008/0004588 A1
(43) Pub. Date: Jan. 3, 2008

(54) Perspiration absorbent pads for female breasts

(76) Inventor: Kathryn Ferrer Gavitt, Cape Coral, FL (US)

Correspondence Address: LAURA G. BARROW, P.O. BOX 215, ESTERO, FL, 33928, US

(21) Appl. No.: 11/480,740

(22) Filed: Jul. 3, 2006

(51) Int. Cl. A61F 13/15

(52) U.S. Cl. 604/385.07; 2/53

(57) ABSTRACT

Various embodiments of perspiration pads for female breasts are provided, which are particularly adapted for placement between the overlying breast and adjacent chest wall to prevent skin-to-skin contact and to absorb perspiration in that area.

### BACKGROUND AND SUMMARY OF THE INVENTION

Contact between the adjacent skin surfaces of the female breast and the chest and torso area are conducive and often create perspiration underneath the female breast. This perspiration trapped within the bra underneath the breast causes discomfort, odor, moisture stains on undergarments and outer garments, rashes, chafing, and in extreme cases dermatological ailments such as intertrigo and yeast infections caused by Candidias albicans. Many women around the world suffer either with simple discomfort and embarrassment or in extreme cases they may suffer from intertrigo or yeast infections that may become further infected, thereby creating open sores underneath the breast. Numerous factors affect the degree of perspiration underneath the breast. Such factors include breast size, obesity, humidity, environmental climate, warm seasonal climate, physical activity, and increase breast size due to pregnancy or nursing. Regardless of which factors create the perspiration, if the perspiration remains trapped underneath the breast, problems as described above begin to occur.

While various pads and the like have been developed to respond to various other perspiration or moisture problems (e.g., underarm shields, sanitary napkins, etc.) none have been developed with enough flexibility to address the needs of numerous varieties of women suffering from breast perspiration. It is acknowledged that other devices have been developed for wear under the breast, but they are purposefully relatively thick and bulky and either intended for cosmetic, uplift purposes. Other pads have been developed to absorb perspiration; however, they are either bulky, address mainly the area between the breasts, or address the entire under area of the breast from underarm to cleavage. Such pads are too bulky and obvious as to not provide discretion of wearing by the user.

A need exists for a perspiration absorbent pad particularly adapted for use by a multitude of women of all shapes and sizes for use in absorbing under breast perspiration. These pads may be placed between the overlying breast and the adjacent chest area, and serve to separate these portions of the anatomy to prevent chafing and other irritation, and as well as serve to absorb perspiration in the immediate area, thereby providing greater comfort and flexibility to women using the pad. The pads may be provided in various embodiments, from relatively thin and flat layered devices, to generally cylindrical, tapered, configuration with or without extending fabric or material to further provide comfort and relief from irritation from the under side of the breast.

The present invention, in certain aspects, is directed to a perspiration absorbent pad for a female comprising an elongated cylindrical portion configured for placement within a brassiere cup. The cylindrical portion is defined in part by a major horizontal axis extending from a lateral end to an opposite medial end of the cylindrical portion. The cylindrical portion further comprises a substantially moisture permeable outer shell and a substantially moisture absorbent inner core surrounded by the outer shell. The pad also includes a bib integral with and extending from the outer shell of the cylindrical portion, the bib oriented parallel to the major axis and configured to extend outside of, and subjacent to, the brassiere cup. The outer shell may be formed of a paper material, for example, and the bib formed of a moisture absorbing material generally known by those of ordinary skill in the art.

Other aspects of the present invention include a perspiration absorbent pad for a female comprising a substantially flat portion configured for placement within a brassiere cup, the flat portion defined in part by a major horizontal axis extending from one a lateral end to a medial end of the flat portion. The flat portion further has an outer shell and a substantially moisture absorbent inner layer surrounded by the outer shell, the flat portion further having a lower edge for alignment above the lower edge of a brassiere cup. The outer shell includes an exterior side formed of a moisture-impermeable material and an interior side formed of moisture-permeable material, the interior side configured for placement against the female breast to absorb moisture therethrough, and the exterior side configured for placement upon an inner surface of the brassiere cup to prevent moisture from penetrating the brassiere cup. The pad also includes a bib integral with, and extending from the lower edge of the outer shell of the flat portion, the bib oriented parallel to the major axis and configured to extend outside of, and subjacent to, the brassiere cup. The exterior side of the outer shell of the pad may further include an adhesive suitable for securing the flat portion of the pad to the inner surface of the cup. In certain embodiments, the flat portion may have a substantially elliptical (i.e. oval) shape or a substantially trapezoidal shape. In still other embodiments, the flat portion may further include a raised cylindrical component formed of moisture absorbing material positioned parallel to the major axis and near the lower edge of the flat portion of the pad to further separate and provide uplift support to the female breast.

Other aspects of the present invention include a perspiration absorbent pad for a female breast comprising a major horizontal axis extending from a lateral side to an opposing medial side of the pad. The pad includes an outer shell and a substantially moisture absorbent inner layer surrounded by the outer shell, the pad further having a lower edge for alignment above the lower edge of a brassiere cup. The outer shell includes an exterior side formed of a moisture-impermeable material and an interior side formed of a moisture-permeable material, the interior side configured for placement against the female breast to absorb moisture therethrough, and the exterior side configured for placement upon an inner surface of the brassiere cup. The medial side further has a segment located near the lower edge of the flat portion which extends therefrom to cover a medial side of the breast when worn within the brassiere. The pad further includes a bib integral with, and extending from, the lower edge of the outer shell of the pad, the bib positioned parallel to the major axis and configured to extend outside of, and subjacent to, the brassiere cup.

The present invention is also directed to methods of absorbing perspiration emanating from a female breast, to thereby prevent chaffing and or infection resulting therefrom while a brassiere is worn. The methods comprise placing the perspiration absorbent pads described herein within a bottom end of a brassiere cup, the cup having a lower edge positioned between the breast and adjacent chest wall of the female, such that the bib of the pad extends completely outside of the brassiere cup below the breast and against the chest wall. Other aspects of the present invention include a method of absorbing perspiration emanating from a female breast when a brassiere is not worn, comprising placing a perspiration absorbent pad between the breast and the chest wall of the wearer, the pad having a (a) moisture-permeable outer shell, wherein the entire outer shell is formed of a moisture-permeable material, and (b) a moisture-absorbent inner layer enclosed within the outer shell, thereby allowing moisture to be absorbed from both the breast and chest wall of the wearer.

### BRIEF DESCRIPTION OF THE DRAWINGS

FIG. 1 is a front view of the first embodiment of the inventive pad, with a portion of the outer shell removed to show the inner core.

FIG. 2 is a sectional view taken along lines 2-2 of FIG. 1.

FIG. 3 illustrates the pad of FIG. 1 inserted within the cups of a brassiere.

FIG. 4 is a sectional side view taken along lines 4-4 of FIG. 3.

FIG. 5 is a perspective view of a brassiere incorporating the pads illustrated in FIGS. 1-4.

FIG. 6 is a side view of a second embodiment of the inventive pad placed beneath the breast.

FIG. 7 is a front elevation of the pad shown in FIG. 6, with a portion of the outer shell removed to show the inner layer.

FIG. 8 is a sectional view taken along lines 8-8 of FIG. 7.

FIG. 9 is a perspective view of a brassiere incorporating the pads illustrated in FIGS. 6-8.

FIG. 10 is a side view of a third embodiment of the inventive pad placed beneath the breast.

FIG. 11 is a front elevation of the pad shown in FIG. 10, with a portion of the outer shell removed to show the inner layer.

FIG. 12 is a sectional view taken along lines 12-12 of FIG. 11.

FIG. 13 is a perspective view of a brassiere incorporating the pads illustrated in FIGS. 10-12.

FIG. 14 is a side view of a fourth embodiment of the inventive pad placed beneath the breast (brassiere not shown).

FIG. 15 is a front elevation of the pad shown in FIG. 14, with a portion of the outer shell removed to show the inner layer.

FIG. 16 is a sectional view taken along lines 16-16 of FIG. 15.

FIG. 17 is a perspective view of a brassiere incorporating the pads illustrated in FIGS. 14-16.

FIG. 18 is a front elevation of a fifth embodiment of the inventive pad.

FIG. 19 is a front elevation of the pad shown in FIG. 18, with a portion of the outer shell removed to show the inner layer.

FIG. 20 is a sectional view taken along lines 20-20 of FIG. 19.

FIG. 21 is a perspective view of a brassiere incorporating the pads illustrated in FIGS. 18-20.

FIG. 22 is a side view of a sixth embodiment of the inventive pad placed between the breast and chest wall.

FIG. 23 is a front elevation of the pad shown in FIG. 22, with a portion of the outer shell removed to show the inner layer.

FIG. 24 is a sectional view taken along lines 24-24 of FIG. 23.

### DETAILED DESCRIPTION OF THE EMBODIMENTS:

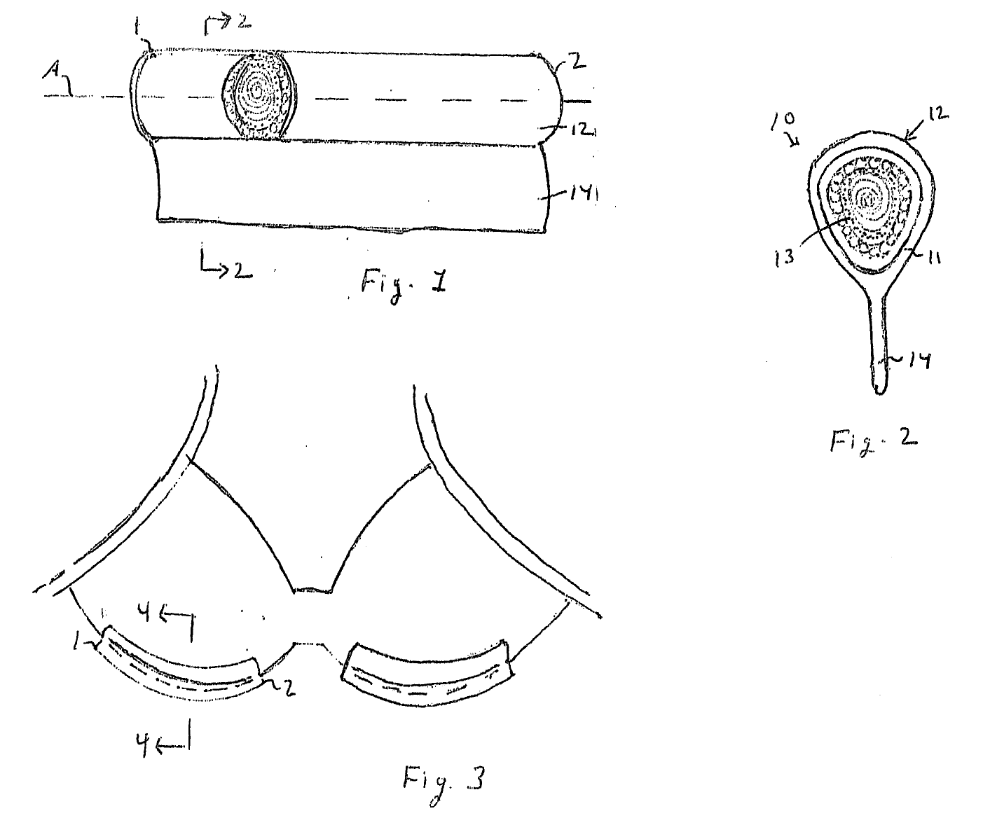

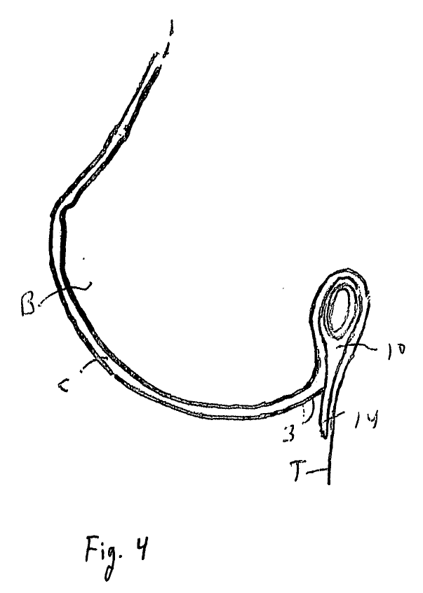

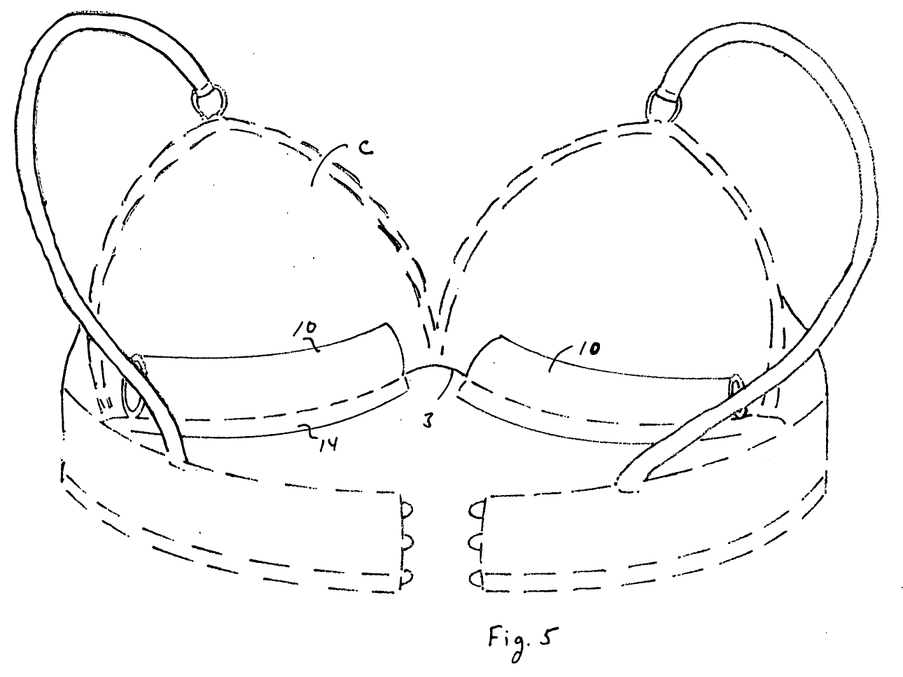

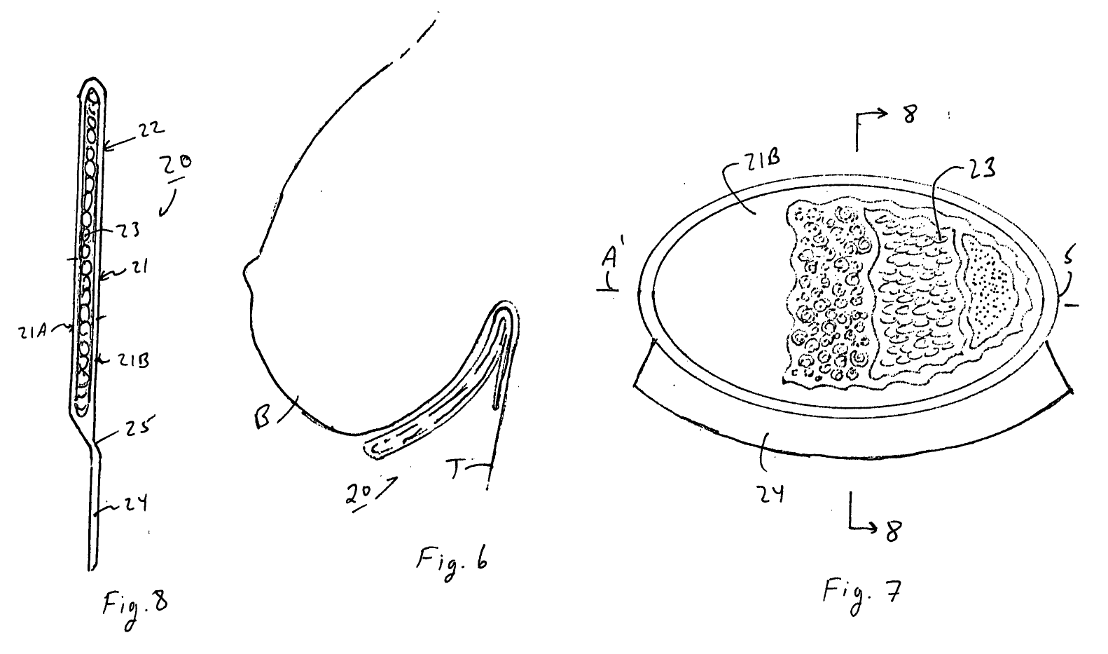

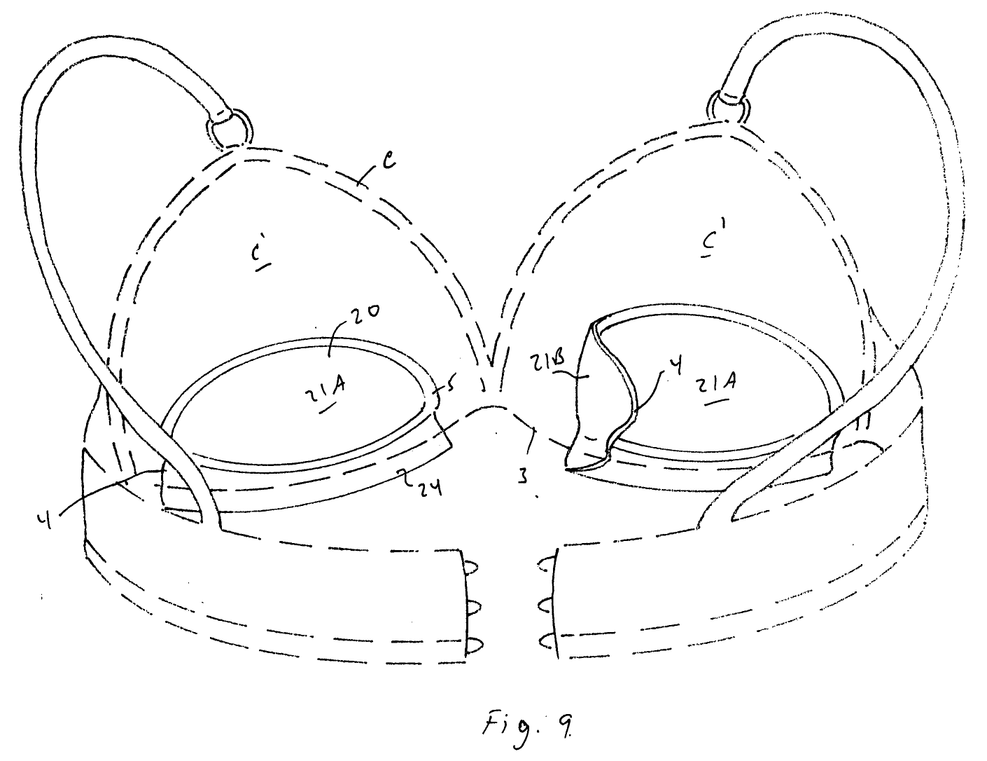

Referring now to the figures, the present invention is directed to feminine perspiration pads and methods of absorbing perspiration emanating from a female breast B, to thereby prevent chaffing and or infection resulting therefrom while a brassiere is worn. FIGS. 1-5 illustrate one embodiment of the inventive pad 10. In this embodiment, the pad comprises an elongated cylindrical portion 11 configured for placement within a brassiere cup C. As shown in FIG. 1, the cylindrical portion 11 is defined in part by a major horizontal axis A extending from the lateral end 1 to the medial end 2 of the cylindrical portion 11.

The cylindrical portion 11 comprises a substantially moisture permeable outer shell 12 and a substantially moisture absorbent inner core 13 surrounded, and thereby encased within, the outer shell 12, as best shown in FIGS. 1-2. Exemplary materials for forming the outer shell are known by those of ordinary skill in the art and include, but are not limited to, paper, cotton, nonwoven materials, and other wicking materials, including those described in the inventor's U.S. Pat. No. 5,603,653 (hereinafter the “'653 patent”), which is incorporated herein by reference in its entirety. Exemplary materials for forming the moisture-absorbent inner core are also known by those of ordinary skill in the art and include, but are not limited to, cotton, paper, non-woven materials, cellulose, as well as those materials described in the '653 patent.

The pad 10 also includes a bib 14 that is integral with, and extends from, the outer shell 12 of the cylindrical portion, as best shown in FIGS. 1-2, and 4. The bib 14 is oriented parallel to the major axis A, as best shown in FIG. 1, and configured to extend outside of, and subjacent to, the cup C of the brassiere (see FIG. 5). By being placed beneath the lower edge 3 of the cup C, in particular where the lower edge consists of an underwire (not shown), the bib 14 functions to provide a buffer to thereby prevent chaffing of the underside of the breast B and chest wall T by the underwire or lower cup section of the brassiere. Optionally, the pad may include a layer adhesive applied to the outer shell to prevent slippage of the pad within the brassiere.

FIGS. 6-9 illustrate another embodiment of the inventive perspiration absorbent pad 20. In this embodiment, the pad comprises a substantially flat portion 22 configured for placement within a brassiere cup (FIG. 9), the flat portion having a lower edge 25 aligned above the lower edge 3 of the brassiere cup C, as shown in FIGS. 8 and 9, for example (in FIG. 6, the cup is not shown for ease of illustration). The flat portion is defined in part by a major horizontal axis A¹ extending from the lateral end 4 to the medial end 5 of the flat portion of the pad, as shown in FIG. 7. The flat portion includes an outer shell 21 and a moisture absorbent inner layer 23 enclosed within the outer shell. The outer shell has an exterior side 21B formed of a moisture impermeable material. In use, the pad is placed within the brassiere as shown in FIG. 9 such that the exterior side 21B of the flat portion of the pad is placed against the inner surface C¹ of the cup C. The moisture impermeable material may be any suitable material for preventing perspiration emanating from the breast from contacting, and consequently penetrating through, the brassiere. Exemplary moisture impermeable materials include, but are not limited to, plastic and other similar polymers, including those described in the '653 patent. Optionally, the exterior side 21B of the outer shell may include an adhesive, lace, or textured fabric to prevent slipping, to allow the pad to be secured firmly within the brassiere cup C, specifically the inner surface C¹ of the cup as shown, thereby minimizing slippage of the pad therein.

The outer shell of the embodiment illustrated in FIGS. 6-9 is also comprised of an inner side 21A formed of a moisture permeable material. The pad is placed within the brassiere cup such that the inner side 21A will press against the breast B. Thus, the moisture permeable material will allow perspiration emanating from the breast to penetrate the inner side 21A to be absorbed within the inner layer 23 formed of the moisture-absorbent material. As discussed above, the exterior side 21B will prevent moisture from exiting the pad, thereby keeping the brassiere and outer clothing dry. Exemplary moisture-permeable materials for fabricating the inner side 21A include, but are not limited to, cotton, mesh, hydrophobic material, non-woven absorbent materials, and the like. Exemplary moisture-absorbent materials for fabricating the inner layer 23, but are not limited to, cotton and cellulose. It will be readily recognized by the skilled artisan, however, that any suitable material capable of absorbing perspiration or permeable to moisture may be employed, including those described in the '653 patent.

The pad shown in FIGS. 6-9 further includes a bib 24 similar to the bib 14 described for the first pad 10 described above. The bib 24 is integral with, and extends from, the lower edge 25 of the outer shell 22. The bib 24 is oriented parallel to the major axis A¹ of the flat portion and configured to extend outside of, and subjacent to, the lower edge 3 of the brassiere cup C to prevent moisture from penetrating the brassiere cup, as described above.

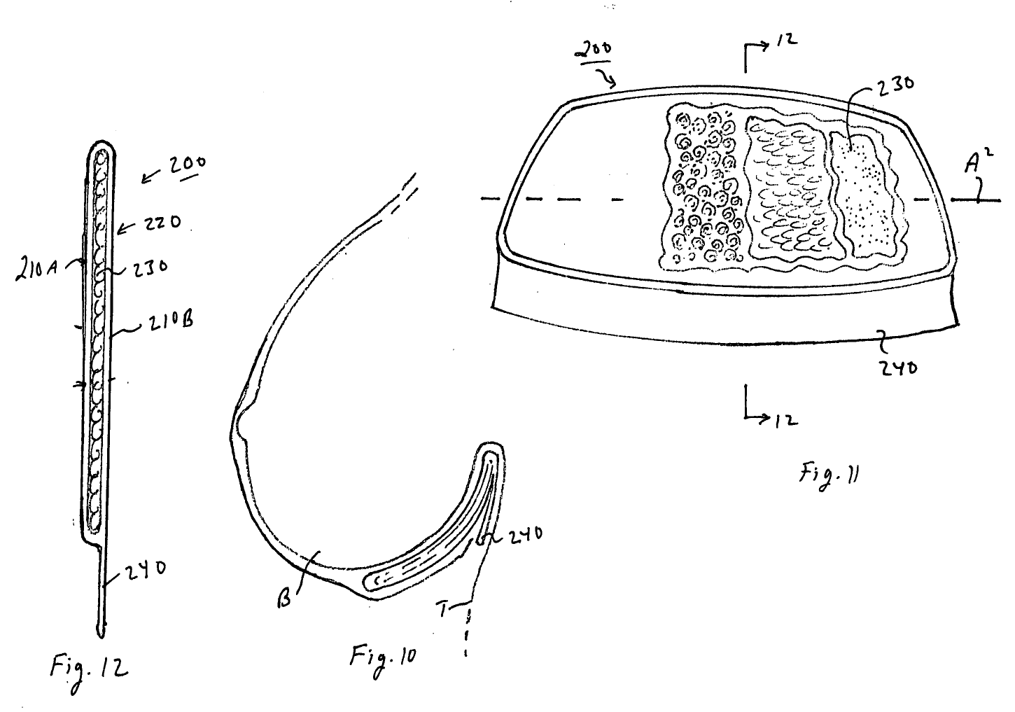

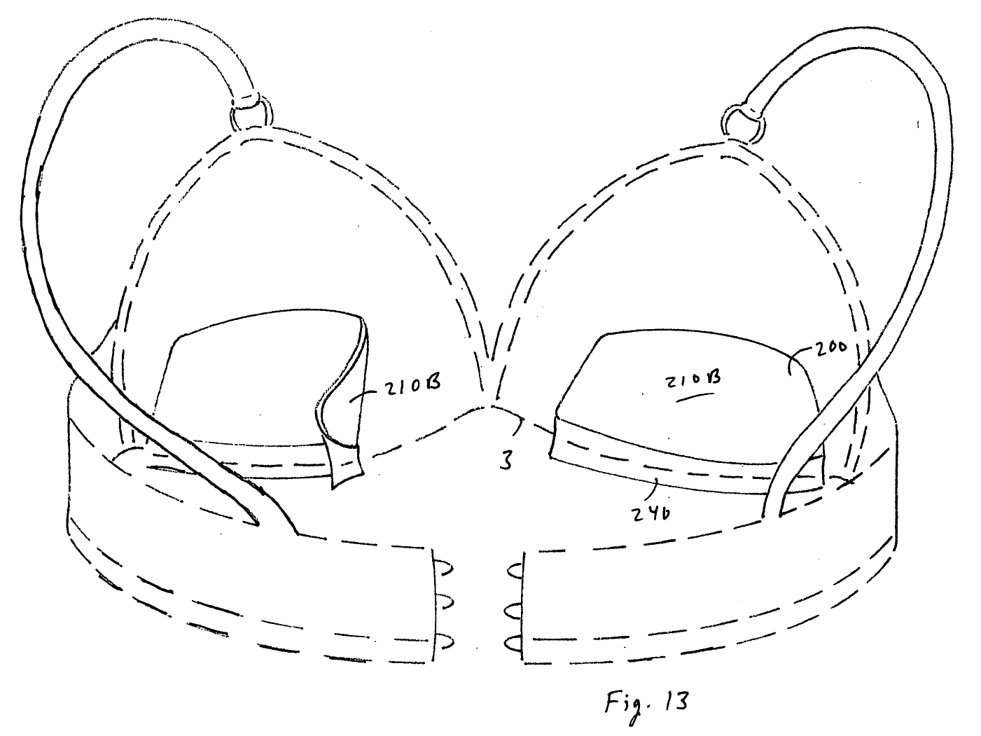

FIGS. 6-9 illustrate the inventive pad 20, wherein the flat portion 21 of the pad has a substantially elliptical shape. This embodiment is particularly well-suited for use in underwire cup bras. FIGS. 10-13 illustrate a pad 200 identical to pad 20 in all aspects except that the shape of the flat portion 210 is substantially trapezoidal (i.e. the base portion is wider than the top portion as best shown in FIG. 11), and is particularly well-suited to soft cup bras. Other features of the pad 200 remain the same, such as the outer shell 220 having an exterior side 210B and an inner side 210A formed of a moisture impermeable material and a moisture-permeable material respectively, an inner moisture absorbent layer 230, a bib 240, and a major axis A², for example. Similarly, the exterior side 210B of the outer shell may include an adhesive that will allow the pad to be secured firmly within the brassiere cup C, thereby minimizing slippage of the pad therein.

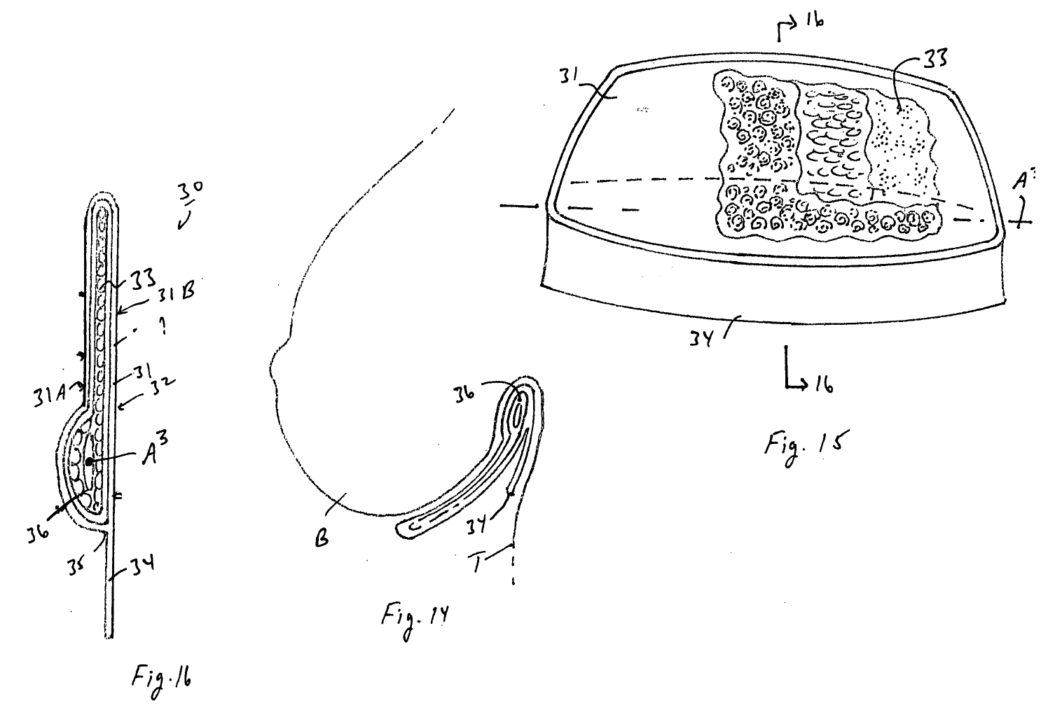

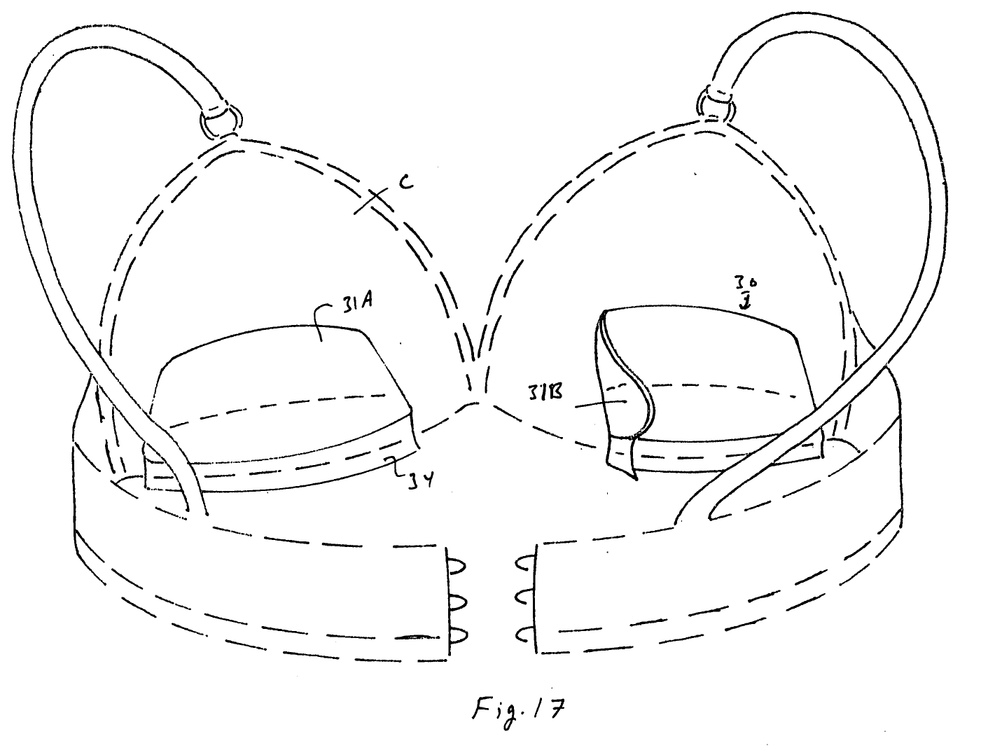

FIGS. 14-17 illustrate another embodiment of the inventive pad 30 which is similar to pad 200, for example, but having a raised cylindrical component 36 formed of a moisture absorbing material which is oriented parallel to the major axis A³ of the flat portion 31 of the pad. This raised cylindrical component 36 functions to provide increased moisture absorption into the pad 30 as well as to further separate and provide uplift and support of the breast. As shown in FIGS. 14 and 16, the cylindrical portion 36 is located near the lower edge 35 of the pad's flat portion 31. The cylindrical portion may be formed of the same or similar moisture absorbing materials described above for the other embodiments of the present invention, and consequently, may be formed of the same or different moisture absorbing material as the remaining inner layer 33 of the pad. In addition, many of the other features of the pads illustrated in FIGS. 6-13 are present in this embodiment, such as the outer shell 32 of the flat portion 31 having an exterior side 31B and an inner side 31A formed of a moisture impermeable material and a moisture-permeable material respectively, the inner moisture absorbent layer 33 discussed above, and a bib 34, for example. Note that for ease of illustration, the brassiere cup is omitted from FIG. 14. Moreover, as for the pads illustrated in FIGS. 1-13, the, the exterior side 31B of the outer shell may include an adhesive that will allow the pad to be secured firmly within the brassiere cup C, thereby minimizing slippage of the pad therein.

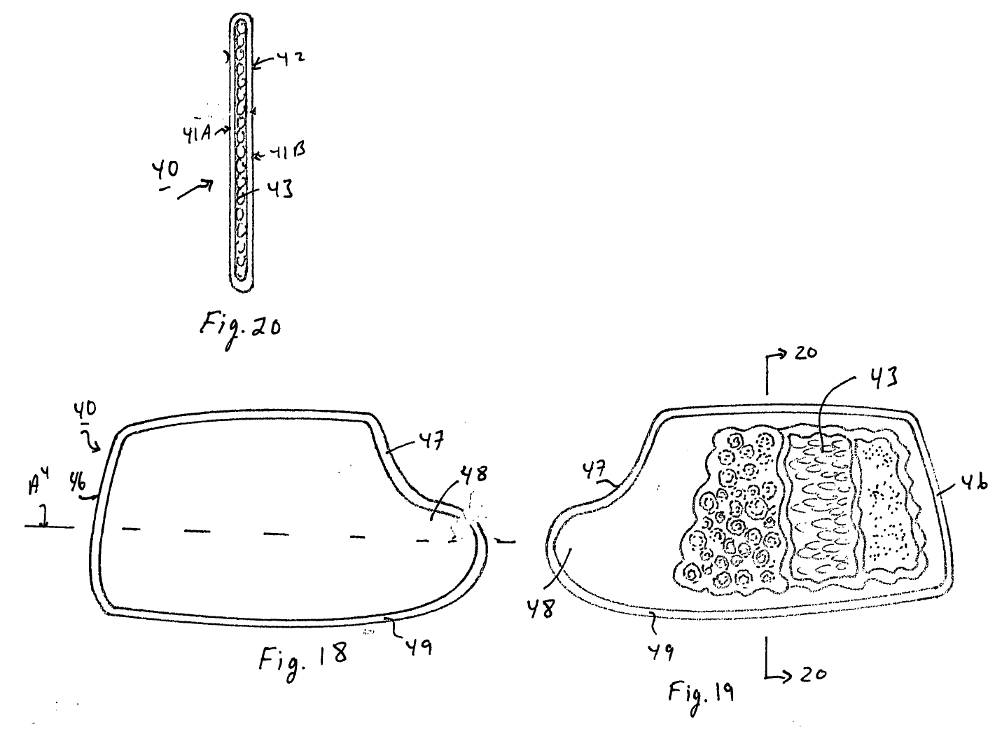

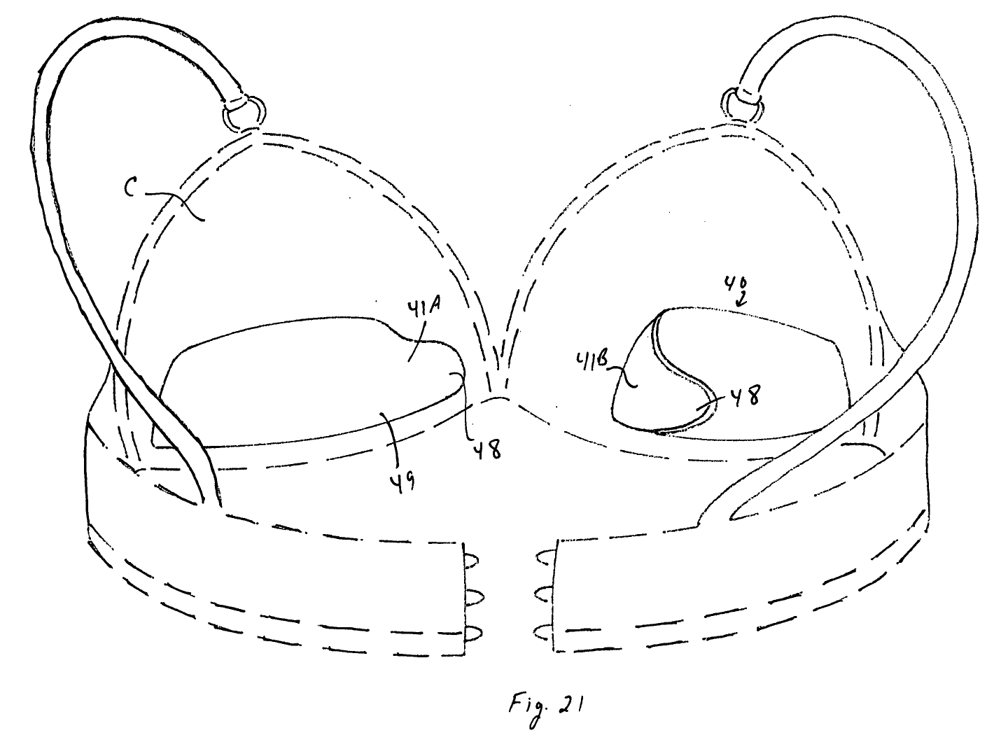

FIGS. 18-21 illustrate another embodiment of the inventive pad 40. Many of the features of this pad 40 are the same as those features described for pads 20, 200. Such feature include the outer shell 42 of the flat portion 41 having an exterior side 41B and-an inner side 41A formed of a moisture impermeable material and a moisture-permeable material respectively, and an inner moisture absorbent layer 43, for example. The pad 40 illustrated in FIGS. 18-20, however, is further defined by its lateral side 46 and opposing medial side 47, with a major horizontal axis A⁴ extending from the lateral and medial sides, as best shown in FIG. 18. Extending from the medial side 47 of the pad is a segment 48 located near the lower edge 49 of the pad and extending therefrom to cover a medial side of the breast (not shown) when worn within the brassiere cup C. As for the other embodiments, the outer shell 42 of the pad may include a layer of adhesive, lace, or textured fabric in reusable form which is applied thereto to prevent slippage of the pad 40 within the brassiere. The pad 40 may also include a bib (not shown) as discussed above for the other embodiments and illustrated in FIGS. 1-17.

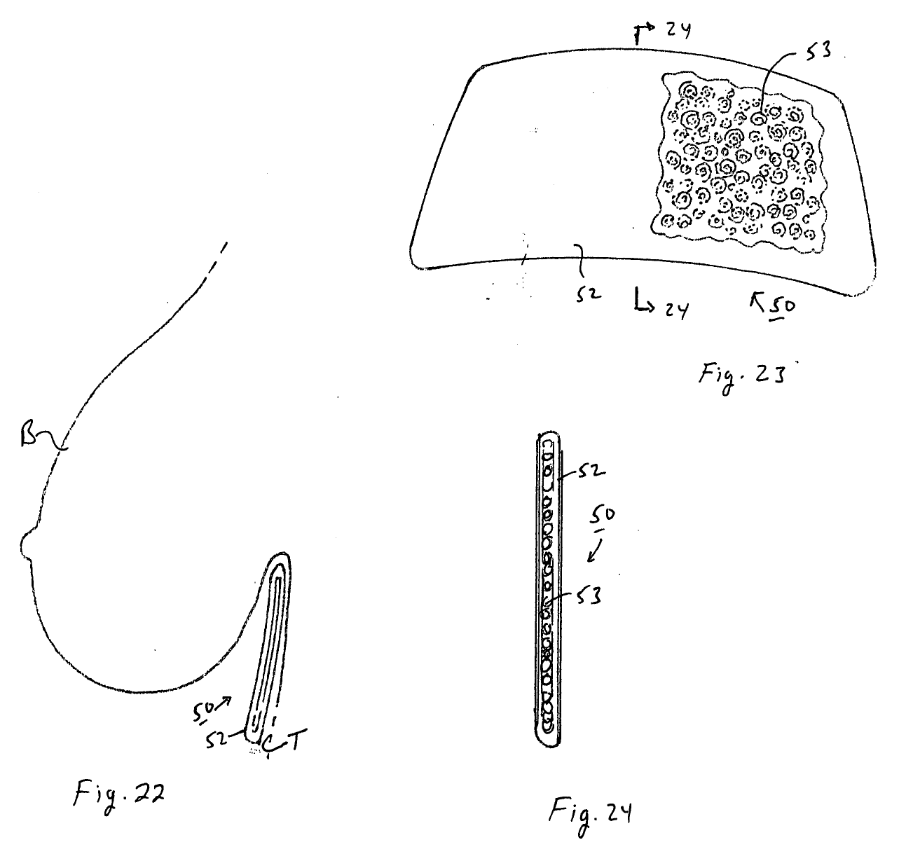

Another embodiment of the present invention is pad 50, illustrated in FIGS. 22-25. This design is particularly useful for obese persons, bedridden patients, or other inactive persons, for example, and is configured to be worn beneath the breast when a brassiere is not worn. The pad 50 comprises an outer shell 52 formed completely of a moisture permeable material. Enclosed completely within the outer shell 52 is an inner layer of moisture absorbent material. Suitable moisture absorbent and moisture permeable materials are the same as for the other pad embodiments described above. The pad 50 is placed between the breast B and chest wall T to absorb perspiration emanating from both the breast B and chest T, as shown in FIG. 22.

I claim:

1. A perspiration absorbent pad for a female, said pad comprising:
a) an elongated cylindrical portion configured for placement within a brassiere cup, said cylindrical portion defined in part by a major horizontal axis extending from a lateral end to an opposite medial end of said cylindrical portion;
b) said cylindrical portion comprising a substantially moisture permeable outer shell and a substantially moisture absorbent inner core surrounded by said outer shell; and
c) a bib integral with and extending from said outer shell of said cylindrical portion, said bib oriented parallel to said major axis and configured to extend outside of, and subjacent to, said brassiere cup.

2. The pad of claim 1, wherein said outer shell if formed of a paper material.

3. The pad of claim 1, wherein said bib is formed of a moisture absorbing material.

4. A method of absorbing perspiration emanating from a female breast, to thereby prevent chaffing and or infection resulting therefrom while a brassiere is worn, said method comprises placing the perspiration absorbent pad of claim 1 within a brassiere cup, said cup having a lower edge positioned between said breast and adjacent chest wall of said female, such that said bib of said pad extends outside of said brassiere cup below said lower end of said cup and said breast and against said chest wall.

5. A perspiration absorbent pad for a female, said pad comprising:
a) a substantially flat portion configured for placement within a brassiere cup, said flat portion defined in part by a major horizontal axis extending from a lateral end to a medial end of said portion;
b) said flat portion having an outer shell and a substantially moisture absorbent inner layer surrounded by said outer shell, said flat portion further having a lower edge for alignment above a lower edge of a brassiere cup;
c) said outer shell having an exterior side formed of a moisture-impermeable material and an interior side formed of moisture-permeable material, said interior side configured for placement against the female breast to absorb moisture therethrough and said exterior side configured for placement upon an inner surface of said brassiere cup; and
d) a bib integral with and extending from said lower edge of said outer shell of said flat portion, said bib oriented parallel to said major axis and configured to extend outside of, and subjacent to, said brassiere cup to prevent moisture from penetrating the brassiere cup and to prevent chaffing.

6. The pad of claim 5, wherein said exterior side of said outer shell comprises an adhesive suitable for adhering said flat portion to said cup.

7. The pad of claim 5, wherein the flat portion has a substantially elliptical shape.

8. The pad of claim 5, wherein the flat portion has a substantially trapezoidal shape.

9. The pad of claim 5, wherein said flat portion further includes a cylindrical component of moisture absorbing material positioned parallel to said major axis and near said lower edge of said flat portion to further separate and provide uplift support to said female breast.

10. A method of absorbing perspiration emanating from a female breast, to thereby prevent chaffing and or infection resulting therefrom while a brassiere is worn, said method comprises placing the perspiration absorbent pad of claim 5 within a brassiere cup, said cup having a lower edge positioned between said breast and adjacent chest wall of said female, such that said bib of said pad extends outside of said brassiere cup below said lower edge of said cup and said breast and against said chest wall.

11. A perspiration absorbent pad for a female breast, said pad comprising:
a) a major horizontal axis extending from a lateral side to an opposing medial side of said pad;
b) an outer shell and a substantially moisture absorbent inner layer surrounded by said outer shell, said pad further having a lower edge for alignment above a lower edge of a brassiere cup;
c) said outer shell having an exterior side formed of a moisture-impermeable material and an interior side formed of moisture-permeable material, said interior side configured for placement against the female breast to absorb moisture therethrough and said exterior side configured for placement upon an inner surface of said brassiere cup; and
d) said medial side further having a segment located near said lower edge of said flat portion and extending therefrom to cover a medial side of said breast when worn within said brassiere.

12. The pad of claim 11, further including a bib integral with and extending from said lower edge of said outer shell of said pad, said bib positioned parallel to said major axis and configured to extend outside of, and subjacent to, said brassiere cup.

13. A method of absorbing perspiration emanating from a female breast, to thereby prevent chaffing and or infection resulting therefrom while a brassiere is worn, said method comprises placing the perspiration absorbent pad of claim 11 within a brassiere cup, said cup having a lower edge positioned between said breast and adjacent chest wall of said female, such that said bib of said pad extends outside of said brassiere cup below said lower edge of said cup and said breast and against said chest wall.

14. A method of absorbing perspiration emanating from a female breast when a brassiere is not worn, said method comprising placing a perspiration absorbent pad under the breast of the wearer, said pad having (a) a moisture-permeable outer shell, wherein said entire outer shell is formed of a moisture-permeable material, and (b) a moisture-absorbent inner layer enclosed within said outer shell, thereby allowing moisture to be absorbed from the breast.

15. The method of claim 14, wherein said pad is placed between the breast and chest wall of the wearer, thereby allowing said moisture to be absorbed from both the breast and chest wall.
